(19) United States
(12) Patent Application Publication
(10) Pub. No.: US 2002/0197611 A1
(43) Pub. Date: Dec. 26, 2002

(54) Method for real-time detection and quantification of nucleic acid sequences using fluorescent primers

(76) Inventor: Alexander Michael Chagovetz, Salt Lake City, UT (US)

Correspondence Address: Susan D. Campbell, Esq., Holme Roberts & Owen, LLP, Suite 4500, 1700 Lincoln, Denver, CO, 80203, US

(21) Appl. No.: 09/886,313

(22) Filed: Jun. 21, 2001

(51) Int. Cl. C12Q 1/68; C12P 19/34

(52) U.S. Cl. 435/6; 435/91.2

(57) ABSTRACT

The invention is a homogeneous method for detecting and/or quantifying specific nucleic acid sequences (DNA or RNA) in amplification or reverse transcription reactions, involving the use of fluorescently labeled forward and reverse primer pairs that produce a fluorescence resonance energy transfer signal when they are incorporated into the complementary strands of a double-stranded reaction product. The invention includes selecting a target sequence and forming the members of the primer pair so that their fluorescent labels are in close proximity, so producing a detectable signal on receiving an energy stimulus. The invention also involves selecting target sequences within short nucleotide fragments, resulting in short amplification products in polymerase chain reactions characterized by relative short duration, high sensitivity and reduced secondary non-specific reactions.

The Assignee, under Rule 37 CFR 1.27, for this non-provisional application is a small entity.

### FIELD OF THE INVENTION

The present invention involves the use of fluorescence including fluorescence resonance energy transfer in polymerase chain reactions to evaluate the identity and quantity of amplified product in a target sample. More specifically, the invention involves forming primer pairs including forward and reverse primers labeled with donor and acceptor flourescent dyes, and utilizing such primer pairs with short target sequence fragments in a polymerase chain reaction to form the double stranded polynucleotide amplification product of the reaction and also, through fluorescent resonance energy transfer between the donor and acceptor fluorescent dyes, to quantitatively evaluate formation of the amplification product. As an alternative, the invention further involves the use of short target sequence fragments and unlabeled primers to produce ultra-short amplification products, the products identified by fluorescent dyes that bind to the amplification products.

### BACKGROUND OF THE INVENTION

The polymerase chain reaction (“PCR”) is a known biotechnology tool. It is used as a necessary preliminary step in DNA and RNA technologies to amplify a few copies of a target sequence to the amounts adequate for typical analytical methods. Real-time quantitative PCR (QPCR) evolved from PCR technology to monitor amplification of a specific target sequence during the progression of PCR.

Fluorescent dye labels typically are small organic dye molecules, such as fluorescein, Texas red, or rhodamine, which can be readily conjugated to probe-type molecules. The fluorescent molecules (fluorophores) can be detected by illumination with light of an appropriate frequency. Light excites the fluorophores and produces a resultant emission spectrum that can be detected by electro-optical sensors or light microscopy.

Fluorescent resonance energy transfer (FRET) occurs between a donor fluorophore and an acceptor dye, which may be a fluorophore, when the donor fluorophore has an emission spectrum that overlaps the absorption spectrum of the acceptor dye, and the donor fluorophore and acceptor dye are in sufficiently close physical proximity. When light excites the donor fluorophore, there is then produced an emission of light that may be absorbed and quenched by the acceptor molecule. When quenching occurs, the intensity of the donor fluorophore's emission appears to be lessened. Where the acceptor is also a fluorophore, the intensity of its fluorescence may be enhanced. The efficiency of energy transfer is highly dependent on the distance between the donor and acceptor, and equations predicting these relationships have been developed by Forster. FRET is a function of the distance between the donor and acceptor molecules. A discussion of these relationships and Forster—type equations is found in K. Parkhurst and L. Parkhurst, Donor—Acceptor Distance Distributions in a Double-Labeled Fluorescent Oligonucleotide both as a Single Strand and in Duplexes, 34 Biochemistry 1995 pp. 293-300.

Various methods have been developed for detecting and quantifying specific sequences of DNA and RNA in the context of polymerase chain reactions. One of the most sensitive methods involves the use of FRET. A common approach is to employ FRET techniques with probe technology to detect and monitor DNA amplification.

One such approach, known as the TaqMan™ assay (Applied Biosystems, Foster City, Calif.; Roche Molecular Systems, Alameda, Calif.), uses a hybridization probe labeled with donor fluorophore and acceptor dye, which is then cleaved by the 5′ to 3′ exonuclease activity of the enzyme Taq polymerase to cause an increase in the intensity of the donor fluorophore. The probe is labeled with both donor and acceptor, and prior to the attachment of the probe to a DNA strand the fluorescence of the donor is quenched by the acceptor. During PCR, the probe is hybridized to the DNA strand to be amplified. As the DNA polymerase acts, the 5′ to 3′ exonuclease activity of the polymerase causes cleavage of the probe, separating the donor and acceptor and resulting in an increase in intensity of the fluorescence of the donor fluorophore. See, for example, Tony Woo, B. K. C. Patel, et al, Identification of Pathogenic Leptospira by TaqMan Probe in LightCycler, 256 Analytical Biochemistry 132-34 (1998).

Another method for detecting amplification products is the “molecular beacon probe.” This method uses oligonucleotide hybridization probes that form hairpin structures, with the donor fluorophore on the 5′ end and the acceptor molecule on the 3′ end of the hybridization probe. When the probe is in the hairpin conformation, the donor and acceptor are in close proximity, and the fluorescence of the donor fluorophore is quenched. During PCR, the molecular beacon probe hybridizes to one of the strands of the PCR product, and is in “open conformation” such that the donor fluorophore and acceptor dye are separated and the fluorescence intensity of the donor increases to a level that can be detected. See Sanjay Tyagi and Fred R. Kramer, Molecular Beacons: Probes that Fluoresce upon Hybridization, 14 Nature Biotechnology 303-08 (1996).

In a further method, PCR may be carried out with a primer labeled with the fluorophore Cy5™ in the presence of a fluorescein-labeled probe. The Cy5™-labeled primer is attached to the target sequence and is used to form an extension product. The fluorescein-labeled probe then hybridizes to the extension product, the Cy5™-labeled strand. When the labeled probe hybridizes to the extension product, the fluorophores of the primer and probe are in close proximity, and resonance energy transfer occurs between the fluorophores, increasing the fluorescence of the Cy5™. In this case, observation of fluorescence and, in particular, the FRET signal, allows monitoring of the hybridization of probe to target, and melting of the probe away from the target, during melting curve analysis of the probe away from the target. See M. J. Lay and C. T. Wittwer, Real-time Fluorescence Genotyping of Factor V Leiden during Rapid Cycle PCR, Clinical Chemistry 43:12 (1997).

The methods described above rely upon the efficiency of the hybridization of the probe to the target. If the probe does not efficiently hybridize to the target sequence, the intensity of the generated signal, used to measure the quantity of the amplification product, is affected. Additionally, the probes may interfere with the DNA amplification process. When a probe binds to the template strand of DNA, it converts a piece of single-stranded template into double-stranded helix. The probe blocks the DNA polymerase from completing translocation along the DNA strand halting replication until the probe is removed, either by melting or enzyme action.

A method which relies not upon probes but only upon applying fluorescent dye labels to a single oligonucleotide primer molecule in a hairpin structure has been described as an approach for detecting the presence of a target nucleic acid sequence and the quantity of such nucleic acid sequence in a sample. In this approach, the primer is designed to have two dye labels on the stem of its hairpin structure. One label is a fluorophore donor and the other is a quencher that absorbs energy emitted by the donor. When the primer molecule is in the hairpin conformation, the fluorophore donor and acceptor are in close proximity, so that the fluorescence of the donor is substantially quenched by the acceptor. Once the primer is attached to the target sequence and replication occurs, the hairpin structure is linearized, a complementary strand is synthesized, and the primer with its fluorescent labels is incorporated into the amplification product. Once the primer is opened and incorporated into the product, the fluorophore donor and acceptor become widely separated, reducing the quenching effect. An advantage of this method is that the fluorescent signal is generated by the product itself, and not through the use of a probe. The method does not measure the amplification product, however, and the use of a hairpin primer involves several difficulties, including design of an awkward, long primer having a hairpin configuration, that may not be easily “read” by DNA polymerase, and the need to place two labels, donor and acceptor, on the hairpin primer in specific locations. The hairpin primer may present other disadvantages, including competition of formation of the hairpin with formation of double-stranded DNA (resulting in lower sensitivity in detection of the FRET signal) and potential formation of primer-dimers, which may interfere with detection of the product signal. Also, the stability of the hairpin has to be low enough to allow enzymatic read through, but high enough to reduce background fluorescence, which creates an inherent contradiction that may reflect on sensitivity of the assay.

### SUMMARY OF THE INVENTION

The present invention provides a method for detecting and quantifying specific nucleotide sequences on a real-time basis in the context of a polymerase chain reaction. The invention involves the identification of target sequences in short deoxyribonucleotide (hereinafter referred to as “nucleotide”) fragments that in turn produce short double-stranded amplification products in the course of PCR. In one embodiment, the invention involves forming one or more primer pairs, each primer pair having a forward and a reverse primer, which are employed in PCR amplification of one or more target sequences, but which are also tagged with fluorescent donor and acceptor dye moieties that generate an FRET signal (or a similarly detectable group) when the forward and reverse primers and dyes of a specific primer pair are brought in close proximity in the PCR product molecule. In a preferred embodiment, the invention involves placing fluorescent dyes on or near the 3′ end of each primer of the primer pair. In PCR, the member primers hybridize to specific target nucleotide sequences, with the result that, where the member primers are incorporated into a double stranded (ds) nucleic acid (DNA) product, their fluorescent dyes are in close proximity and generate a characteristic FRET signal. In one aspect, the invention involves measuring, on a real time basis, the intensity of the FRET signal to directly determine the quantity of ds DNA amplification product in the sample volume. In another aspect of the invention, the intensity of the signal can be used to calculate the number of copies of the target sequence in the original sample. In yet another aspect, the intensity of the signal indicates the distance between the members of the primer pair, which may correlate to specific occurrences within the original target sequence such as the length of a deletion/insert area. In yet another aspect of the invention, the invention includes a reverse transcription step, using at least one member of the primer pair of the invention, preliminary to the polymerase chain reaction. The method of employing fluorescently-tagged primer pair in PCR, the primers structured to hybridize to single stranded target nucleotide sequences and produce a short ds nucleotide amplification product with the members of the primer pair incorporated in the product in close proximity, generating an FRET signal, is utilized to great advantage in various applications, which are described in greater detail below. In an alternative embodiment, the primers are not labeled with dyes before PCR commences, but fluorescent dyes are utilized in solution where PCR takes place, the dyes binding to the short double stranded amplification product molecules as they are formed, producing a characteristic signal.

### BRIEF DESCRIPTION OF THE DRAWINGS

FIG. 1 depicts a possible primer structure according to the invention.

FIG. 2 depicts the method of one embodiment of the invention as applied to DNA amplification.

### DETAILED DESCRIPTION

The present invention involves a method for detecting and quantifying a target nucleic acid sequence in nucleic acid amplification, using primers labeled with fluorescent dyes. The invention may be applied to DNA or RNA target sequences. An initial step involves forming a target nucleotide sequence. In a preferred embodiment, the target nucleotide sequence is located in a short nucleotide fragment, its length preferably being in the range of no less than about 25 to no more than about 100 nucleotides in length. This initial step includes forming the short nucleotide fragment, which will in turn form a short amplification product. As will be discussed below, the use of short nucleotide fragments to produce short amplification products supports the use of dyes having an FRET relationship and results in various distinct advantages in PCR, including speed and reduced risk of side reactions.

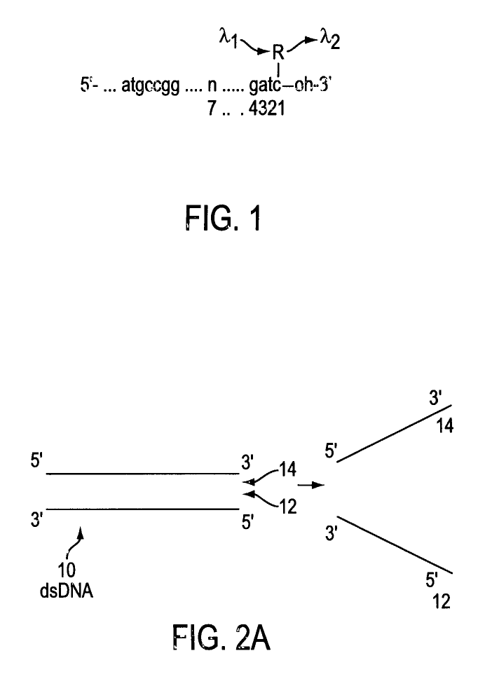

The invention includes forming a primer pair, in which the two members of the pair are forward and reverse primers, each primer including a nucleotide sequence that binds, or anneals, to a preferred site on the target sequence, and that serves as a point of initiation for a primer extension reaction wherein the primer is extended in the 5′ to 3′ direction. The invention includes forming the primers to anneal to the selected target sequence in a location that results in the two primers of the primer pair being only a short distance apart, within a specific proximity, in the amplification product formed as a result of a polymerase chain reaction. In a specific embodiment, the invention further involves labeling each member of the primer pair with a fluorescent dye, the fluorescent dyes of each primer pair being spectrally overlapping and interacting to produce an FRET signal or similar detectable signal when the fluorescent dyes are within a specific proximity and are subject to an energy stimulus, such as exposure to light of a particular wavelength. In a preferred embodiment, each primer of the primer pair is structured as an oligonucleotide, in the range of about 10 to about 40 nucleotides long, with a free (reactive) 3′-OH functionality, and a phosphodiester or otherwise modified (peptide, phosphotiol) backbone, and having a fluorescent or otherwise detectable label on one of the one to seven nucleotides from the 3′ end. As depicted in the diagram of FIG. 1, the primer structure of this embodiment includes a sequence of nucleotides, having a 5′ end and 3′ end, with the —OH functionality at the 3′ end. The fluorescent label R is attached proximal to the 3′ end, connected by a linker chain to a cytosine nucleotide on one of the first to seventh nucleotides from the 3′ end. When light of wavelength λ₁ is applied to the primer, the label R is excited, and subsequently emits light of a different wavelength λ₂. The primers of a primer pair of the present invention are formed to hybridize to target sequences in a location that results in the two primers being a short distance apart in a double stranded DNA amplification product. The fluorescent label is preferably attached to a thymine or cytosine nucleotide, with a linker chain between the dye and the cytosine or thymine nucleotide. In a preferred embodiment, the gap between the primers when incorporated in the amplification product does not exceed a distance of about 10 base pairs, the distance between the fluorescent dyes on such incorporated primers preferably does not exceed about 100 Angstroms (A°), and the amplification product does not exceed a length of about 130 base pairs, with the most preferred length of the amplification product being in the range of about 25 to about 100 base pairs. Accordingly, the length of the fragment containing the target sequence also is not in excess of about 130 nucleotides, and has a preferred length in the range of about 25 to about 100 nucleotides. It will be appreciated that these distances may vary as a function of specific dyes used, measuring device sensitivity, nucleotide chain structure, and other factors. In an alternative embodiment, the invention involves first forming short target sequence fragments of the length described above, containing the selected target sequence. The embodiment then involves forming a primer pair, coordinated with the target sequence, that is not labeled with dyes preliminary to PCR. Rather, a dye is introduced into the PCR solution, with the primers, and other reaction components, prior to the reaction. The dye then binds to the double stranded amplification products as they are formed in solution and the characteristic signal of the dye is measured as the reaction progresses. The dye is an intercalating fluorescent dye, also called a minor groove binding dye, which exhibits fluorescence upon binding to a double stranded amplification product on direct excitation with light. Examples of dyes used for this purpose include SYBR Green (Molecular Probes, Oregon) and ethidium bromide.

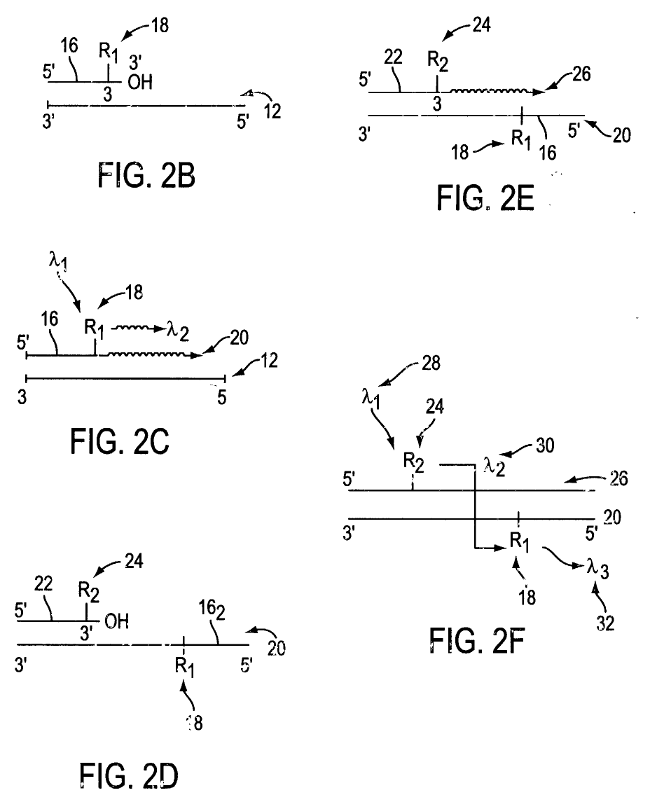

In a preferred embodiment, the invention is applied within the context of amplification of a nucleic acid target sequence through a polymerase chain reaction in a closed reaction system. The reaction is preferably conducted in a sealed or capped transparent capillary vial within an instrument that performs thermal cycling needed for PCR as well as fluorescence acquisition, such as the Roche LightCycler™. As depicted in FIG. 2A, in the initial stage of the reaction, heat is applied to a sample containing a small number of copies of double stranded nucleic acid, which nucleic acid contains the selected target sequence. During this step, the double stranded nucleic acid molecules 10 of the sample melt, or denature, to form two single strands 12, 14. In the present example, the nucleic acid is DNA. The reaction solution includes a quantity of forward and reverse primer pairs, formed and fluorescently labeled according to the invention, along with a quantity of a thermostable nucleic acid polymerase, such as Taq DNA polymerase, and a quantity of deoxynucleotidetriphosphates, all in an aqueous reaction medium including an appropriate buffer, and magnesium chloride. The first (forward) primer 16, with its donor fluorophore dye label 18, is structured to attach to the specific target nucleotide sequence on a single DNA strand 12. As depicted in FIG. 2B, the forward primer 16 hybridizes to the target sequence of the single DNA strand 12, and serves as a starting seed for replicating a complement to the target DNA strand, the target strand 12 serving as a template for replication. As depicted in FIG. 2C, the primer 16 is extended along the DNA template strand by the DNA polymerase, to form a new strand of DNA 20. The forward primer 16 and its fluorescent dye label 18 are thereby incorporated into the new strand of DNA 20. As the amplification reaction progresses, melting occurs again, producing a pool of single-stranded DNA products with fluorescent dyes, including the new strand 20. The unattached reverse primer molecule 22, with its acceptor fluorophore dye label 24, in the reaction solution then hybridizes to a target sequence on the new single strand 20 with incorporated forward primer 16 and attached fluorescent dye 18, as depicted in FIG. 2D. The reverse primer 22 is then utilized by DNA polymerase to build a new strand of DNA 26 complementary to the strand 20 incorporating the tagged forward primer 16, see FIG. 2E. As depicted in FIG. 2F, the resulting double stranded DNA product, comprised of the strands 20 and 26, has a fluorescent tag incorporated on each strand. When light of a first wavelength λ₁ 28 is shown on the reaction mixture, the donor fluorophore 24 experiences excitation, and subsequently emits light of a second wavelength λ₂ 30 which is absorbed by the acceptor fluorophore 18, the acceptor fluorophore 18 then emitting light of a third wavelength λ₃ 32. In a preferred embodiment, the fluorescent dyes have been placed at the 3′ end of the primers used to create the extension product, and the dyes are then accordingly located on the internal sequences of the complementary DNA strands. The target sequence fragments and primers are formed to produce resultant amplification product strands that are short, preferably not more than about 130 base pairs in length, depending on the characteristics of the fluorescent dyes, enabling the fluorescent dyes of the incorporated primers to engage in FRET “cross talk.” The close proximity of the tags in the amplification product permits FRET signals to be generated on excitation of the donor fluorophore found on one primer of the original pair. The FRET signals are then measured optically or otherwise instrumentally, such as by a spectrofluorimeter. Preferably, the application of an energy stimulus, such as application of light, and measurement of the resulting signal, such as an FRET signal, all occurs within a single instrument that is also used for the thermal cycling required for PCR.

As the PCR progresses, and the amount of double labeled amplification product in the reaction volume increases, the intensity of the FRET signal increases. In a preferred embodiment, the invention includes real-time monitoring of the concentration of the amplification product in the reaction volume, and the progress of the polymerase chain reaction, as a function of the intensity of the FRET signal. Preferably, the invention includes a control experiment, involving the use of water, preferably deionized water, instead of target nucleic acid sequence, to determine the background level of fluorescence. As the concentration of the amplification product increases, the intensity of the FRET signal increases above the background level and is detected by the monitoring equipment.

In one embodiment of the invention, the method includes labeling the forward and reverse primers with donor and acceptor fluorophores respectively. In this embodiment, when the donor and acceptor fluorophores are brought in close proximity in an amplification product, and light of a first wavelength is shown on the reaction system, the donor fluorophore is excited and then produces an emission of energy at a second wavelength. The acceptor fluorophore then becomes excited and absorbs energy at the second wavelength, subsequently producing an emission of energy, ordinarily at a third wavelength. This emission of energy by the acceptor fluorophore is then detected by a spectrofluorimeter or other similar measuring device, when the quantity of amplification product and donor-acceptor fluorophore pairs in close proximity reaches a level above the characteristic background level established through a control reaction. As will be appreciated by those familiar with the field, the placement of donor versus acceptor fluorophores on forward and reverse primers can be reversed, and it is also possible to measure other varieties of FRET signals, such as the “quenching” effect that occurs between a donor fluorophore and acceptor dye which is not a fluorophore but only an acceptor that absorbs and quenches the energy emitted by the donor fluorophore on excitation.

It should also be noted that alternate embodiments of the invention involve the use of other types of dye pairs, such as luminescent, phosphorescent or otherwise detectable groups, in place of fluorescent labels on the primers of the primer pair. Such alternate dyes operate to interact when brought into close proximity, and then, following an energy stimulus, produce a signal. Such alternate labels or tags may interact through any mechanism that produces a detectable signal when brought within a specific proximity in the double stranded amplification product. As will be recognized, use of varying signal producing labels can affect the location of the labels on the primers, the structure or length of the primers, the selection of the target sequence, and the specific proximity of the labels in the amplification product necessary to permit a detectable signal to be generated when a doubly labeled amplification product is formed. Also, in an alternative embodiment, the primer pairs are not initially labeled with dyes before PCR, but the method includes introducing a fluorescent dye in the reaction system which binds to the double stranded amplification product as it is formed in PCR.

The use of a primer-only system (as opposed to probe-only or probe-primer systems) for generating FRET signals or other detectable signals enhances the efficiency of DNA amplification. The primers are directly utilized in amplification. Probes may interfere with the amplification process, blocking the action of DNA polymerase as it seeks to extend the primer along the template strand. In addition, it is presently believed that there may be an inefficiency in the hybridization of probes to the correct target sequences (they may miss the target), which reduces the reliability of FRET as a direct indicator that amplification is occurring. Moreover, there may be a relatively long waiting time for probes to hybridize to the target sequences (sometimes 15-40 seconds), which provides an opportunity for side reactions.

In a preferred embodiment of the invention, relatively short segments of target nucleic acid (in the range of about 25 to about 100 nucleotides) are amplified, and the time needed for amplification is accordingly short. In an embodiment using fluorescently-labeled primers, the primer pairs of the present invention are designed to hybridize to target sequences such that the FRET signal is produced when the double-stranded amplification product, incorporating a fluorescently tagged primer pair, is formed. A preferred embodiment of the invention involves forming primer pairs in which the fluorescent label attached to each member of the pair is located within about one to seven nucleotides from the 3′ end of the primer, on a cytosine (C) nucleotide. In a further embodiment, the target sequence and the primer sequences are selected so that the distance between the forward and reverse primer binding sites is small, less than about ten nucleotides. When the primers and their fluorescent labels are separated by a minimal distance in the double stranded amplification product, the intensity of the FRET signal is high, and the FRET signal will generally appear above the background threshold early in the PCR reaction, when a relatively small quantity of amplification product has been formed. There is a direct correlation between the intensity of the FRET signal and the quantity of double stranded amplification product within the system. Also, when the target sequence is relatively short, and the system does not rely on probes to generate the FRET signal (which require extended times for hybridization) or a possibly awkward hairpin-style primer, the time for the reaction is relatively short, and monitoring of the progress of the PCR is highly sensitive, with reduced inefficiency due to side reactions (there is little time for side reactions to occur).

An alternate embodiment of the present invention includes design of specific discriminating primer pairs intended to amplify a very narrow target region. This embodiment includes selective amplification of target sequences on the background of similar but not identical sequences, such as pseudo genes, and species variations. In another embodiment, the invention includes co-amplifying similar sequences as distinctly different products for quantification over a wide dynamic range. Primer pairs for different target sequences are structured, each primer pair having different donor and acceptor fluorescent tags, such that ds DNA products of specific target sequences are formed with distinctly different FRET signals and effects.

In a further embodiment, the invention includes a reverse transcription step preliminary to polymerase chain reaction. The reverse transcription step occurs in the same closed reaction vessel as PCR. This step includes introducing messenger RNA (mRNA) templates into the reaction vessel, with deoxynucleotide triphophates, DNA polymerase, and fluorescently-tagged primers designed to attach to the single mRNA strands. An initial reverse transcription cycle with the forward primer of the primer pair and ordinary thermostable DNA polymerase produces strands of DNA complementary to each mRNA strand (cDNA) with the mRNA molecule serving as a template. Reverse primer formed according to the invention then hybridizes to the cDNA strands, and DNA polymerase then extends the primer along the cDNA strand to form ds DNA. The primers are designed to hybridize to target sequences on the template mRNA strands and the cDNA strands, so that the product ds DNA segments are relatively short. The primers are fluorescently tagged with donor and acceptor dyes, which, once incorporated into the ds DNA, produce an FRET signal as amplification of the cDNA occurs. Because the mRNA segment to be used for transcription is short, the reverse transcription step occurs in a relatively short time period, and ordinary thermostable polymerases, such as Taq polymerase and PFU (a thermostable DNA polymerase similar to Taq but originating from a different bacterial source), can be used to perform the reverse transcription step, instead of thermally sensitive viral reverse transcriptase. The same polymerase can be used throughout the reaction, including PCR in which the reaction volume is heated to melt ds DNA and then amplify it.

In another embodiment, the primer pairs of the present invention are employed in mutation analysis, such as analysis of single nucleotide polymorphisms. In one approach, the primer pair is formed so that the mutation spot on a target sequence coincides with the 3′ end of one of the primers. Once the first primer hybridizes to the strand over the mutation spot, DNA polymerase extends the primer to form a new complementary strand. The new ds DNA is then melted, to form single stranded DNA. The second primer of the pair then hybridizes to the new strand at a point such that the area of the new strand complementary to the mutation spot becomes a template for the second strand of the ds DNA product. The two primers are fluorescently tagged with donor and acceptor moieties. The primers are structured to hybridize to the target sequence and are incorporated into the ds DNA product, generating an FRET signal. The primer pair thus encompasses the target sequence including a mutation point. It is possible using primer pairs to identify samples containing mutations, and to quantify the amount of mutated nucleic acid in the sample as a function of fluorescent intensity produced by the FRET interaction of the amplification product.

Additionally, in another embodiment, the invention involves providing a method for the identification of two or more variants simultaneously. A first primer pair (fluorescently tagged, with a specific FRET relationship and signal) amplifies a first variant on a DNA (RNA) strand. A second primer pair (also fluorescently tagged, with a different specific FRET relationship and signal) amplifies a second variant on the DNA (RNA) strand. The two primer pairs thus amplify, identify and quantify two mutation points, simultaneously, in the same reaction vessel. The method includes measuring instrumentally the two different FRET signals simultaneously. As will be apparent, the present invention also includes forming multiple primer pairs, for amplifying short target sequences containing mutations, so permitting analysis of clustered (multiple) single nucleotide polymorphisms within a short span of DNA.

The invention further includes a method for measurement of the length of a deletion/insertion area. The invention involves structuring the primer pair to hybridize sequences at the outer edges of the deletion/insertion area, so straddling the deletion/insertion. Each primer of the pair is tagged at or near its 3′ end, and the primer is formed to hybridize to target strands at points of rearrangement under interrogation so that the distance between the donor and acceptor dyes approximates the length of the deletion/insertion. The FRET signal intensity is a function of the distance between the donor and acceptor dyes. The FRET signal intensity accordingly indicates the length of the deletion/insertion. This approach provides significant advantages when analyzing degenerate sequences of DNA, such as di- or tri-nucleotide repeats. A primer pair is structured to flank the degenerate sequence, and the length of the sequence (number of repeats) equals the distance between the primer fluorescent dyes (again, the fluorescent tags are at or near the 3′ end) in the PCR product, which correlates to the intensity of the FRET signal. In yet another embodiment, the primer pairs of the present invention are used to detect and quantify gross rearrangements of DNA sequence, by amplification of the sequences at the break point/junction of the rearrangements. In the case of a single gross rearrangement, with two break points, a primer pair is designed to encompass, and amplify, each break point sequence. Again, measurement of the FRET signal's characteristics indicates presence of a mutation, and quantity in the original sample.

In another embodiment of the invention, melting analysis may also be applied as a further step following completion of amplification. Double-stranded DNA molecules have characteristic thermal stabilities, which are usually expressed in terms of the melting temperatures. While melting temperatures of longer (˜>80 bp) ds DNA tend to converge in the range of 90°-94° C., the shorter fragments retain characteristic “signature” melting temperatures (Tm), which serves as one of the methods to identify the DNA fragment or distinguish between similar DNA fragments (polymorphic DNA). The dsDNA Tm's are currently used for mutation analysis with sequence-specific DNA probes: molecular beacons, hybridization probes, etc. The technology of the present invention produces extremely short amplification products as compared to traditional PCR. This enables us to apply melting analysis for discrimination between specific and non-specific amplification products, as well as to distinguish between polymorphisms (single nucleotide polymorphisms and short deletions/insertions in the amplicon sequence.) The advantage of using the double labeled short DNA products is that there is no competition between probes and same sense strand DNA for hybridizing with the target sequence. This leads to potentially significantly higher fluorescence intensity, hence higher sensitivity. The fact that we directly observe the melt of the amplicon (and not the probe-amplicon hybrid) allows us to convert melting analysis from a qualitative to a quantitative analytical technique. There are several distinctly different cases for using thermal stability to discriminate between different variants of the target DNA.

1. The polymorphic region is located in the sequence between the primers. Amplification of both sequences should proceed with similar efficiencies (in a vast majority of cases), which generates a similar (indistinguishable) fluorescence signal. However, during the melt we should observe several melting events, corresponding to differences in the amplicon's sequences. Relative changes of fluorescence intensity during each of the melts (relative amplitude of the signal) should be directly correlated with the amount of the particular DNA variant in the PCR products.

2. Discriminating acquisition of the fluorescence signal. If different amplification products (as described above) have different thermal stabilities, we can perform fluorescence acquisition at relatively high temperatures, which allows us to selectively monitor specific (high melting) products in the real time mode, while amplification of the less stable products remains fluorescently silent. This application is of utmost importance for excluding low melting non-specific amplification products (primer dimers) from generating detectable signal (solution for the false positive sample identification). Alternatively, in the case of the target DNA polymorphisms, we can apply low temperature acquisitions for monitoring the “group amplification” of different species in real time, and resolving them by post-amplification melting analysis (see above).

3. Polymorphic positions are spanned by one (or both primers). If the SNP (or short deletion/mutation) position is spanned by the sequence(s) of proposed primers (not at the ends of the primers), their thermal stabilities on the target sequence may be dramatically different due to destabilisation of the hybrid by the mismatches. We can design the temperature profile of PCR to either perform a consensus amplification (corresponding to the lowest stability of the primer/target hybrid), or to perform selective amplification of the most stable species (high annealing temperatures) with annealing point higher than the Tm of the lower melting species.

### EXAMPLE

The example that follows illustrates various aspects of the present invention but is not intended to limit in any way its scope as more particularly set forth in the claims. The following example involves the formation of fluorescently labeled primers according to the method of the invention for quantification of the RNA sequence for human NF-E2 transcription factor (GenBank accession no. XM006816). The invention involves the application of a polymerase chain reaction in a closed reaction system to a first step of reverse transcription of an mRNA target sequence and then to amplification of the cDNA strand formed by reverse transcription. This application involves formation of primer pairs which each include a forward primer and a reverse primer, the primers each being tagged with fluorescent labels that interact to produce a fluorescent signal (FRET) when the labels are brought within a specific proximity. It should be noted that this proximity varies according to the type of labels used, the detection equipment, and other factors. What follows is a general overview of the sequence of this application.

In the first cycle of the application, the forward primer with a fluorescent label is applied to a single strand messenger RNA (mRNA) sample target sequence with a thermostable DNA polymerase in a reverse transcription step to produce a strand of DNA complementary to the mRNA strand (cDNA). In the next cycle, which occurs in the same closed reaction system, the mRNA/cDNA double-stranded molecule is melted, and the fluorescently labeled reverse primer then hybridizes to the cDNA strand. The thermostable DNA polymerase then extends the reverse primer to form a new strand along the cDNA template. The double-stranded product incorporates both the forward primer and the reverse primer, with their fluorescent labels. The mRNA target sequence and primer structures are selected so that in the double-stranded amplification product, which incorporates both the forward and reverse primers with their fluorescent labels, the labels are in close proximity and an FRET signal is detectable after a sufficient quantity of the double-stranded product is formed. A control reaction is performed to determine the background level of fluorescence.

In the example, the method of the invention first involves identifying a mRNA target sequence and then forming forward and reverse primer nucleotide sequences coordinated with the target sequence. This is ordinarily accomplished by a software program such as GenBank-Entrez. The target sequence of the cDNA derived from the mRNA is as follows: AGCACCTTCGGGATGAATCAGGCAACAGCTACTCTCCTG. The forward primer for the cDNA amplification is an oligodeoxynucleotide having a nucleotide sequence: 5′-AGCACCTTCGGGATGAATC-3′. The reverse primer for use with cDNA amplification is an oligodeoxynucleotide having the following nucleotide sequence: 5′-CAGGAGAGTAGCTGTTGCC-3′. The distance between the nucleotide binding sites on the target sequence is one nucleotide. The fluorescent dyes applied to label the primers are Oregon Green 488 for the forward primer and Alexa 633 for the reverse primer, both dyes having been obtained from Molecular Probes, Inc., of Eugene, Oreg. The synthesis of the primers includes standard phosphoramidate solid phase synthesis including use of an amino-modifier—deoxyribocytidine (dC)—CPG at the 3′ end of the primer. The Oregon Green 488 and Alexa 633 dyes, in the form of succynilimide (NHS) esters, are attached to the forward and reverse primers, respectively, at the primary amino group at the 3′ end deoxyribocytidines (dC's) by NHS-primary amino conjugation, according to the manufacturer's protocols. The 3′ OH groups are left free, and are the starting points for polymerization. The resulting labeled primers are purified by reverse phase high pressure liquid chromatography (RP/HPLC) using a TEAA-acetonitrile solvent system. The primers are then lyophilized, resuspended in deionized water, and the solution divided into 10 microliter doses having a primer concentration in solution of about 5 micromoles per liter.

The monitoring of fluorescence emission during PCR and reverse transcription thermal cycling were both performed in the LightCycler™ (Roche). The reaction vessel for both the reverse transcription step and polymerase chain reaction is a capped, optically transparent capillary vial, recommended for use with the LightCycler™. The total reaction volume is about 10 microliters. The reaction constituents include an initial quantity of mRNA target sequence (which can range from 1 to 10⁶ initial copies), a 1 microliter aliquot of aqueous primer solution as described above, an aliquot of 1 microliter of aqueous reaction medium including standard aqueous Roche HP buffer (10×), a 1200 microMolar concentration of deoxynucleotide triphosphates and a concentration of about 3 mM magnesium in the form of magnesium chloride (MgCl₂), and a quantity of DNA polymerase, preferably Roche standard Taq polymerase (or Hotstart™), in the amount of about 0.4 units (manufacturer's recommendations) per reaction. The particular dyes chosen are detected by the Roche Light Cycler™, with acquisition in Channels 1 and 2 of the LightCycler™. The reaction includes a first reverse transcription step (one cycle), followed by about 45 cycles of PCR amplification. During each cycle the temperature is initially brought to 94° C. (0 seconds), which causes the double-stranded target molecules to melt forming single stranded nucleic acids. Once the temperature reaches 94° C., it is then dropped at the maximum rate (20° C. per second per the manufacturer's programming) to approximately 60° C. in a ramping down process that lasts approximately 5 seconds. As the temperature drops the primers then begin to anneal, with an expected approximately 50% of the primers annealing to target at the low point temperature of 60° C. It is expected that amplification occurs also during this relatively short reaction format. The temperature is not lowered further in order to avoid nonspecific primer-dimer type side product reactions. The temperature is then raised to 75° C. for fluorescence acquisition, in a ramping process that occurs for approximately 2 seconds and then to 94° C. to begin the next cycle. The same conditions are applied to each of about 45 cycles of amplification, which follow the initial reverse transcription cycle. The reverse transcription and PCR cycles occur in the same closed vessel. Since the donor and acceptor dyes attached to the forward and reverse primers are separated by only 1 nucleotide when incorporated into the amplification product, there will be a high signal intensity at a relatively low concentration of amplicons, and, on applying the LightCycler Channel 2 acquisition, an FRET signal characteristic of the specific dye pair is expected to emerge above baseline at about 35 cycles of amplification for 10 initial mRNA copies. The signal observed is the emission of the Alexa 633 acceptor fluorophore, as a result of FRET which occurs when both the Oregon Green 488 and Alexa 633 are incorporated into the double stranded amplification product of the cDNA and the Oregon Green 488 is excited by a transmission of light from the LightCycler™. The expected typical signal intensity is proportional to actual DNA concentration.

In negative control experiments, the sample RNA was replaced with sterile water. At the acquisition temperature, 75° C., it is not expected to see any real-time fluorescent signals originating from primer-dimer formation, because possible primer-dimers should have a melting temperature significantly lower than that temperature (usually 50° to 65° C). Primer-dimers could be observed during the melting phase of PCR, for example during a slow (0.1 degree per second) ramp from 40° to 90° C. with concurrent acquisition. After the completion of PCR, the invention includes an optional step of validation of the signal specificity by melting analysis of the double stranded amplicon. Non-specific amplification products, such as primer-dimer products should melt at temperatures significantly lower than those of the amplification products. An additional or alternative (independent) control includes 12% polyacrylamide gel electrophoresis of the amplification reaction products. The reaction products are visualized as fluorescent bands of specific mobility on the gel.

What is claimed is:

1. A method for detecting a nucleic acid target sequence in a sample, comprising the steps of:
(a) forming a nucleic acid target sequence;
(b) forming a primer pair for amplifying said target sequence in a polymerase chain reaction, said primer pair including a forward primer and a reverse primer;
(c) labeling said forward primer with a first dye;
(d) labeling said reverse primer with a second dye, said first and second dyes interacting to produce a signal when they are located within a specific proximity and are subjected to an energy stimulus;
(e) introducing a quantity of said forward and reverse primers, a quantity of thermostable nucleic acid polymerase, a quantity of deoxynucleotide triphosphates, and a quantity of said nucleic acid target sequence in aqueous reaction medium, in a reaction vessel;
(f) initiating a polymerase chain reaction in said reaction vessel to amplify said target sequence, said polymerase chain reaction producing a first quantity of double stranded amplification product of said target sequence, said amplification product incorporating said forward and reverse primers on opposite complementary strands of said amplification product and said first dye of said forward primer and said second dye of said reverse primer located within said specific proximity and producing a signal on application of said energy stimulus, detectable by a measuring device.

2. The method of claim 1, wherein said first and second dyes are fluorescent dyes.

3. The method of claim 1, wherein said first and second dyes are selected from a group that includes luminescent and phosphorescent dye moieties.

4. The method of claim 1, wherein said signal involves fluorescent resonance energy transfer.

5. The method of claim 1, wherein said specific proximity is not greater than about 100 A°.

6. The method of claim 1, wherein said measuring device is a spectroflourimeter.

7. The method of claim 1, wherein said target sequence has a length of up to about 130 nucleotides.

8. The method of claim 1, wherein said target sequence has a length in the range of about 25 nucleotides to about 100 nucleotides.

9. The method of claim 1, wherein said amplification product has a length in the range of no greater than about 130 base pairs.

10. The method of claim 1, wherein said first dye is a donor fluorophore and said second dye is an acceptor fluorophore, and wherein said application of said energy stimulus produces fluorescent resonance energy transfer (FRET) between said first dye and said second dye, and said signal is a fluorescent emission signal produced by said second dye following FRET.

11. The method of claim 1, wherein a control reaction is completed, using water in place of said target sequence, establishing a background signal level which may be compared to said signal produced by said amplification product.

12. The method of claim 1, comprising an additional step of conducting a melting temperature analysis of said amplification product, following said step of initiating a polymerase chain reaction.

13. The method of claim 1, wherein said target sequence includes a mutation point, and said amplification product includes a copy of said mutation point.

14. The method of claim 13, wherein said step of forming said first and second primers includes structuring the nucleotide sequences of said primers to hybridize to nucleotide sequences flanking said target sequence, and wherein said signal is analyzed to determine the length of said target sequence.

15. The method of claim 1, comprising the steps of:
(a) forming a second primer pair for amplifying a second target sequence, said second primer pair including a second forward primer and a second reverse primer;
(b) labeling said second forward primer with a third dye;
(c) labeling said second reverse primer with a fourth dye, said third and fourth dyes interacting to produce a second signal when they are located within a second specific proximity and are subjected to a second energy stimulus;
(d) at the time of said first step of introducing, second introducing a quantity of said second forward and second reverse primers and a quantity of said second target sequence in said aqueous reaction medium in said reaction vessel; and
(e) at the time of said first step of initiating, second initiating a second polymerase chain reaction to amplify said second target sequence producing a first quantity of a second double stranded amplification product of said second target sequence, said second amplification product incorporating said second forward primer and said second reverse primer on opposite complementary strands of said second amplification product, and said third dye and said fourth dye located within a specific proximity and producing a second signal on application of said second energy stimulus, detectable by said measuring device, permitting simultaneous amplification, detection and quantification of two target sequences.

16. The method of claim 1, wherein said target sequence is a sequence of messenger RNA (mRNA), and wherein said step of initiating a polymerase chain reaction includes a first cycle that includes annealing said forward primer to said mRNA target sequence and producing a complementary strand of DNA (cDNA), and includes further cycles amplifying said cDNA strand, the double stranded amplification product incorporating said forward and reverse primers and producing a signal on application of an energy stimulus, detectable by a measuring device.

17. A method for detecting a nucleic acid target sequence in a sample, comprising the steps of:
(a) forming a nucleic acid target sequence having a length of up to 130 nucleotides;
(b) forming a primer pair for amplifying said target sequence in a polymerase chain reaction to produce a double stranded amplification product, said primer pair including a forward primer and a reverse primer;
(c) preparing a dye for labeling said double stranded amplification product in solution when such amplification product is formed;
(d) introducing a quantity of said forward and reverse primers, a quantity of thermostable nucleic acid polymerase, a quantity of deoxynucleotide triphosphates, a quantity of said dye, and a quantity of said target sequence in an aqueous reaction medium;
(e) inducing an amplification reaction, producing a quantity of amplification product derived from said target sequence, said dye thereafter labeling said amplification product; and
(f) detecting said labeled amplification product with a measuring device.

18. The method of claim 17, wherein said nucleic acid target sequence has a length in the range of from about 25 nucleotides to about 100 nucleotides.

19. A method for forming a primer pair, including a forward and reverse primer, for use in nucleic acid amplification, comprising the steps of:
(a) forming a forward primer and a reverse primer, each primer including a sequence of nucleotides with a 3′ end and a 5′ end, and having a reactive —OH functionality on the 3′ end, each said sequence of nucleotides corresponding to a portion of a nucleic acid target sequence to which said primer anneals during amplification; and
(b) labeling each of said forward primer and reverse primer with a member of a fluorescent dye pair, each member of the dye pair connected to said primer at a nucleotide located from one to seven nucleotides from the 3′ end of said sequence of nucleotides, said dye pair members producing a detectable signal when said forward and reverse primers are incorporated into the opposite complementary strands of a double stranded amplification product in which said primers are located within a specific proximity.
